(19) United States
(12) Patent Application Publication
(10) Pub. No.: US 2009/0131778 A1
(43) Pub. Date: May 21, 2009

(54) DEVICES, SYSTEMS, METHODS AND TOOLS FOR CONTINUOUS GLUCOSE MONITORING

(76) Inventors: Arvind N. Jina, San Jose, CA (US); Beelee Chua, Fremont, CA (US); Janet Tamada, Stanford, CA (US); Michael J. Tierney, San Jose, CA (US); Shashi P. Desai, San Jose, CA (US)

Correspondence Address: SHAY GLENN LLP, 2755 CAMPUS DRIVE, SUITE 210, SAN MATEO, CA, 94403, US

(21) Appl. No.: 12/275,145

(22) Filed: Nov. 20, 2008

Related U.S. Application Data
(63) Continuation-in-part of application No. 11/277,731, filed on Mar. 28, 2006.
Application No. 11/277,731 is a continuation-in-part of application No. 11/642,196, filed on Dec. 20, 2006.

(51) Int. Cl. A61B 5/145

(52) U.S. Cl. 600/365

(57) ABSTRACT

One aspect of the invention provides a glucose monitor having a plurality of tissue piercing elements, each tissue piercing element having a distal opening, a proximal opening and interior space extending between the distal and proximal openings; a sensing volume in fluid communication with the proximal openings of the tissue piercing elements; sensing fluid extending into the sensing volume; and a glucose sensor adapted to detect a concentration of glucose in the sensing fluid within the sensing volume. Another aspect of the invention provides A method of in vivo monitoring of an individual's interstitial fluid glucose concentration comprising: inserting distal ends of a plurality of tissue piercing elements through a stratum corneum area of the individual's skin, the tissue piercing elements each comprising a distal opening, a proximal opening, and an interior space extending between the distal and proximal opening; allowing interstitial fluid to flow into the interior space of the tissue piercing elements to substantially fill the interior space; filling substantially the entire interior space of the sensing area; and sensing a glucose concentration of the sensing fluid.

### CROSS-REFERENCE

This application is a Continuation-In-Part of U.S. patent application Ser. No. 11/277,731 filed Mar. 28, 2006 (Publication No. 20060219576). This application is also a Continuation-in-Part of U.S. patent application Ser. No. 11/642,196 filed Dec. 20, 2006 (Publication No. 20080154107).

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The invention relates to systems, devices, and tools, and the use of such systems, devices and tools for monitoring blood glucose levels in a person having diabetes. More specifically, the invention relates to systems, devices, and tools and the use of such systems, devices and tools for monitoring blood glucose level continuously, or substantially continuously.

Diabetes is a chronic, life-threatening disease for which there is no known cure. It is a syndrome characterized by hyperglycemia and relative insulin deficiency. Diabetes affects more than 120 million people world wide, and is projected to affect more than 220 million people by the year 2020. It is estimated that 1 in 3 children today will develop diabetes sometime during their lifetime. Diabetes is usually irreversible, and can lead to a variety of severe health complications, including coronary artery disease, peripheral vascular disease, blindness and stroke. The Center for Disease Control (CDC) has reported that there is a strong association between being overweight, obesity, diabetes, high blood pressure, high cholesterol, asthma and arthritis. Individuals with a body mass index of 40 or higher are more than 7 times more likely to be diagnosed with diabetes.

There are two main types of diabetes, Type I diabetes (insulin-dependent diabetes mellitus) and Type II diabetes (non-insulin-dependent diabetes mellitus). Varying degrees of insulin secretory failure may be present in both forms of diabetes. In some instances, diabetes is also characterized by insulin resistance. Insulin is the key hormone used in the storage and release of energy from food.

As food is digested, carbohydrates are converted to glucose and glucose is absorbed into the blood stream primarily in the intestines. Excess glucose in the blood, e.g. following a meal, stimulates insulin secretion, which promotes entry of glucose into the cells, which controls the rate of metabolism of most carbohydrates.

Insulin secretion functions to control the level of blood glucose both during fasting and after a meal, to keep the glucose levels at an optimum level. In a normal person blood glucose levels are between 80 and 90 mg/dL of blood during fasting and between 120 to 140 mg/dL during the first hour or so following a meal. For a person with diabetes, the insulin response does not function properly (either due to inadequate levels of insulin production or insulin resistance), resulting in blood glucose levels below 80 mg/dL during fasting and well above 140 mg/dL after a meal.

Currently, persons suffering from diabetes have limited options for treatment, including taking insulin orally or by injection. In some instances, controlling weight and diet can impact the amount of insulin required, particularly for non-insulin dependent diabetics. Monitoring blood glucose levels is an important process that is used to help diabetics maintain blood glucose levels as near as normal as possible throughout the day.

The blood glucose self-monitoring market is the largest self-test market for medical diagnostic products in the world, with a size of approximately $3 billion in the United States and $5.0 billion worldwide. It is estimated that the worldwide blood glucose self-monitoring market will amount to $8.0 billion by 2006. Failure to manage the disease properly has dire consequences for diabetics. The direct and indirect costs of diabetes exceed $130 billion annually in the United States—about 20% of all healthcare costs.

There are two main types of blood glucose monitoring systems used by patients: single point or non-continuous and continuous. Non-continuous systems consist of meters and tests strips and require blood samples to be drawn from fingertips or alternate sites, such as forearms and legs (e.g. OneTouch® Ultra by LifeScan, Inc., Milpitas, Calif., a Johnson & Johnson company). These systems rely on lancing and manipulation of the fingers or alternate blood draw sites, which can be extremely painful and inconvenient, particularly for children.

Continuous monitoring sensors are generally implanted subcutaneously and measure glucose levels in the interstitial fluid at various periods throughout the day, providing data that shows trends in glucose measurements over a short period of time. These sensors are painful during insertion and usually require the assistance of a health care professional. Further, these sensors are intended for use during only a short duration (e.g., monitoring for a matter of days to determine a blood sugar pattern). Subcutaneously implanted sensors also frequently lead to infection and immune response complications. Another major drawback of currently available continuous monitoring devices is that they require frequent, often daily, calibration using blood glucose results that must be obtained from painful finger-sticks using traditional meters and test strips. This calibration, and re-calibration, is required to maintain sensor accuracy and sensitivity, but it can be cumbersome as well as painful.

At this time, there are four products approved by the FDA for continuous glucose monitoring, none of which are presently approved as substitutes for current glucose self-monitoring devices. Medtronic (www.medtronic) has two continuous glucose monitoring products approved for sale: Guardian® RT Real-Time Glucose Monitoring System and CGMS® System. Each product includes an implantable sensor that measures and stores glucose values for a period of up to three days. One product is a physician product. The sensor is required to be implanted by a physician, and the results of the data aggregated by the system can only be accessed by the physician, who must extract the sensor and download the results to a personal computer for viewing using customized software. The other product is a consumer product, which permits the user to download results to a personal computer using customized software.

A third product approved for continuous glucose monitoring is the Glucowatch® developed by Cygnus Inc., which is worn on the wrist like a watch and can take glucose readings every ten to twenty minutes for up to twelve hours at a time. It requires a warm up time of 2 to 3 hours and replacement of the sensor pads every 12 hours. Temperature and perspiration are also known to affect its accuracy. The fourth approved product is a subcutaneously implantable glucose sensor developed by Dexcom, San Diego, Calif. (www.dexcom.com). All of the approved devices are known to require daily, often frequent, calibrations with blood glucose values which the patient must obtain using conventional finger stick blood glucose monitors.

### SUMMARY OF THE INVENTION

The invention is a novel continuous glucose monitor that may be periodically calibrated without using finger sticks or other invasive calibration techniques and measures glucose without extracting any interstitial fluid (or any other fluid) from the user. The continuous glucose monitor may be configured to be self-calibrating.

One aspect of the invention provides a glucose monitor with a plurality of tissue piercing elements, each tissue piercing element having a distal opening, a proximal opening and interior space extending between the distal and proximal openings; a sensing area in fluid communication with the proximal openings of the tissue piercing elements; sensing fluid extending from the sensing area into substantially the entire interior space of the tissue piercing elements; and a glucose sensor adapted to detect a concentration of glucose in the sensing fluid within the sensing area. This arrangement permits interstitial fluid glucose to diffuse from the interstitial fluid into the sensing area without extracting interstitial fluid through the distal openings of the piercing elements into the interior space. In some embodiments, the glucose monitor has a removable cover extending over the distal openings of the tissue piercing elements.

In some embodiments, the glucose monitor has a display adapted to display a glucose concentration sensed by the sensor. The display may be disposed within a housing separate from the sensor, with the glucose monitor further including a communicator adapted to wirelessly communicate sensor information from the sensor to the display.

In some embodiments, the glucose monitor includes a sensing fluid reservoir and a pump adapted to move sensing fluid out of the sensing fluid reservoir into the sensing area. Such embodiments may have a manual actuator and may have a waste reservoir adapted to receive sensing fluid from the sensing area. In some such embodiments, the glucose monitor may have a housing with a first part and a second part, the first part of the housing being adapted to support the tissue piercing elements, the sensing fluid reservoir, the sensing area, and at least part of the sensor, the second part of the housing having an electrical connection to the at least part of the sensor in the first part of the housing, with the housing further including a connector adapted to connect and disconnect the first part of the housing from the second part of the housing. In some embodiments, the first part of the housing is further adapted to support the pump and optionally the waste reservoir. Some embodiments have a communicator supported by the second part of the housing and adapted to communicate sensor information to a display.

In some embodiments, the sensing fluid in the sensing fluid reservoir has a glucose concentration of between about 0 mg/dl and about 400 mg/dl. The sensing fluid may also contain buffers, preservatives or other materials in addition to the glucose. In yet other embodiments, the glucose monitor has an adhesive element adjacent the tissue piercing elements and adapted to attach to a user's skin. The glucose sensor, tissue piercing elements and sensing area may be further adapted to detect a concentration of glucose in the sensing fluid within the sensing area without extracting interstitial fluid through the distal openings into the interior space.

Another aspect of the invention provides a method of in vivo monitoring of an individual's interstitial fluid glucose concentration including the following steps: inserting distal ends of a plurality of tissue piercing elements through a stratum corneum area of the individual's skin, the tissue piercing elements each having a distal opening, a proximal opening, an interior space extending between the distal and proximal openings, and a sensing fluid filling substantially the entire interior space; and sensing a glucose concentration of the sensing fluid. This method permits interstitial fluid glucose to diffuse from the interstitial fluid into the sensing area without extracting interstitial fluid through the distal openings of the piercing elements into the interior space. Some embodiments include the step of removing a cover from the distal openings of the tissue piercing elements prior to the inserting step. Some embodiments include the step of displaying glucose concentration information remote from the stratum corneum area of the individual's skin. The method may also include the step of wirelessly communicating glucose concentration information to a display.

In some embodiments, the sensing step is performed by a sensor in fluid communication with a sensing area and the interior spaces of the tissue piercing elements, and the method further includes the step of calibrating the sensor by moving sensing fluid into the sensing area, such as by using a pump. The method may also include the step of moving sensing fluid out of the sensing area as sensing fluid is moved into the sensing area. The sensing fluid may have a glucose concentration of between about 0 mg/dl and about 400 mg/dl.

In embodiments in which the step of moving sensing fluid includes the steps of moving sensing fluid from a sensing fluid reservoir, the sensing fluid reservoir, sensing area, tissue piercing elements and at least part of the sensor may be supported by a first part of a housing, and the method further includes the step of attaching the first part of the housing to a second part of the housing prior to the inserting step, with the second part of the housing having an electrical connection to the at least part of the sensor in the first part of the housing. The method may also include the step of separating the second part of the housing from the first part of the housing after the sensing step.

In some embodiments, the method includes the step of attaching the tissue piercing elements to the individual with adhesive. In other embodiments, the method includes the step of permitting glucose to diffuse from interstitial fluid of the individual through the distal openings into the interior space.

Another embodiment of the invention includes a glucose monitor comprising a plurality of tissue piercing elements, each tissue piercing element comprising a distal opening, a proximal opening and an interior space extending between the distal and proximal openings; a sensing area in continuous fluid communication with the proximal openings of the tissue piercing elements; sensing fluid extending from the sensing area into substantially the entire interior space of the tissue piercing elements; and a glucose sensor adapted to continuously detect a concentration of glucose in the sensing fluid within the sensing area further adapted to be self-calibrating.

Other embodiments of the invention will be apparent from the specification and drawings.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a cross-sectional schematic view of a glucose monitoring device according to one embodiment of the invention in place on a user's skin.

FIG. 2 shows an exploded view of a glucose monitoring device according to another embodiment of the invention.

FIGS. 3(a) and (b) are a schematic representative drawing of a three electrode system for use with the glucose sensor of one embodiment of this invention.

FIGS. 4(a) and (b) are a schematic representative drawing of a two electrode system for use with the glucose sensor of one embodiment of this invention.

FIG. 5 is a cross-sectional schematic view of a portion of a glucose monitoring device according to yet another embodiment of the invention.

FIG. 6 shows a remote receiver for use with a glucose monitoring system according to yet another embodiment of the invention.

FIG. 7 shows a glucose sensor in place on a user's skin and a remote monitor for use with the sensor.

FIG. 8 is a cross-sectional schematic view of a portion of a glucose monitoring device according to yet another embodiment of the invention.

### DETAILED DESCRIPTION OF THE INVENTION

The present invention provides a significant advance in biosensor and glucose monitoring technology: portable, virtually non-invasive, self-calibrating, integrated and non-implanted sensors which continuously indicate the user's blood glucose concentration, enabling swift corrective action to be taken by the patient. The sensor and monitor of this invention may be used to measure other analytes as well, such as electrolytes like sodium or potassium ions. As will be appreciated by persons of skill in the art, the glucose sensor can be any suitable sensor including, for example, an electrochemical sensor an optical sensor.

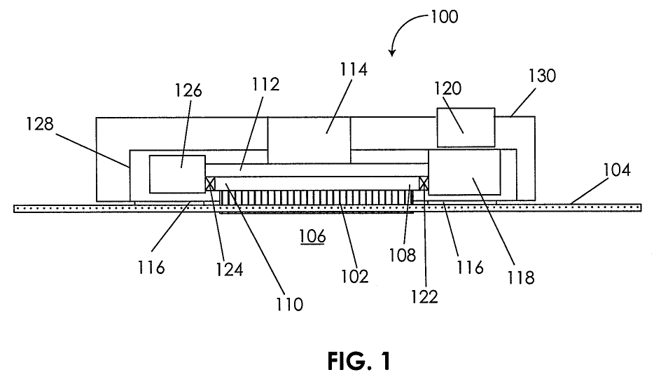

FIG. 1 shows a schematic cross-section of one embodiment of the invention in use. The glucose monitor 100 has an array of unique hollow microneedles 102 or other tissue piercing elements extending through the stratum corneum 104 of a user into the interstitial fluid 106 beneath the stratum corneum. Suitable microneedle arrays include those described in Stoeber et al. U.S. Pat. No. 6,406,638; U.S. Patent Appl. Publ. No. 2005/0171480; and U.S. Patent Appl. Publ. No. 2006/0025717. The needles in array 102 are hollow and have open distal ends, and their interiors communicate with a sensing area 110 within a sensor channel 108. Sensing area 110 is therefore in fluid communication with interstitial fluid 106 through microneedle array 102. In this embodiment, sensing area 110 and the microneedles 102 are pre-filled with sensing fluid prior to the first use of the device. Thus, when the device is applied to the user's skin and the microneedles pierce the stratum corneum of the skin, there is substantially no net fluid transfer from the interstitial fluid into the microneedles. Rather, glucose diffuses from the interstitial fluid into the sensing fluid within the needles, as described below.

Disposed above and in fluid communication with sensor channel 108 is a glucose sensor 112. In some embodiments, glucose sensor is an electrochemical glucose sensor that generates an electrical signal (current, voltage or charge) whose value depends on the concentration of glucose in the fluid within sensing area 110. Details of the operation of glucose sensor 112 are discussed in more detail below.

Sensor electronics element 114 receives the voltage signal from sensor 112. In some embodiments, sensor electronics element 114 uses the sensed signal to compute a glucose concentration and display it. In other embodiments, sensor electronics element 114 transmits the sensed signal, or information derived from the sensed signal, to a remote device, such as through wireless communication. Glucose monitor 100 is held in place on the skin 104 by one or more adhesive pads 116.

Glucose monitor 100 has a novel built-in sensor calibration system. A reservoir 118 containing a sensing fluid having, e.g., a glucose concentration between about 0 and about 400 mg/dl. In some embodiments, the glucose concentration in the sensing fluid is selected to be below the glucose sensing range of the sensor. The sensing fluid may also contain buffers, preservatives or other components in addition to the glucose. Upon actuation of a pump manually or automatically, plunger or other actuator 120, sensing fluid is forced from reservoir 118 through a check valve 122 (such as a flap valve) into sensing channel 108. Any sensing fluid within channel 108 is forced through a second check valve 124 (e.g., a flap valve) into a waste reservoir 126. Check valves or similar gating systems are used to prevent contamination. Because the fresh sensing fluid has a known glucose concentration, sensor 112 can be calibrated at this value to set a base line. After calibration, the sensing fluid in channel 108 remains stationary, and glucose from the interstitial fluid 106 diffuses through microneedles 102 into the sensing area 110. Changes in the glucose concentration from over time reflect differences between the calibration glucose concentration of the sensing fluid in the reservoir 118 and the glucose concentration of the interstitial fluid which can be correlated with the actual blood glucose concentration of the user using proprietary algorithms. Because of possible degradation of the sensor or loss of sensor sensitivity over time, the device may be periodically recalibrated by operating actuator 120 manually or automatically to send fresh sensing fluid from reservoir 118 into sensing area 110.

In some embodiments, microneedle array 102, reservoirs 118 and 126, channel 108, sensor 112 and adhesive pads 116 are contained within a support structure (such as a housing 128) separate from electronics element 114 and actuator 120, which are supported within their own housing 130. This arrangement permits the sensor, sensing fluid and microneedles to be discarded after a period of use (e.g., when reservoir 118 is depleted) while enabling the electronics and actuator to be reused. A flexible covering (made, e.g., of polyester or other plastic-like material) may surround and support the disposable components. In particular, the interface between actuator 120 and reservoir 118 must permit actuator 120 to move sensing fluid out of reservoir 118, such as by deforming a wall of the reservoir. In these embodiments, housings 128 and 130 may have a mechanical connection, such as a snap or interference fit.

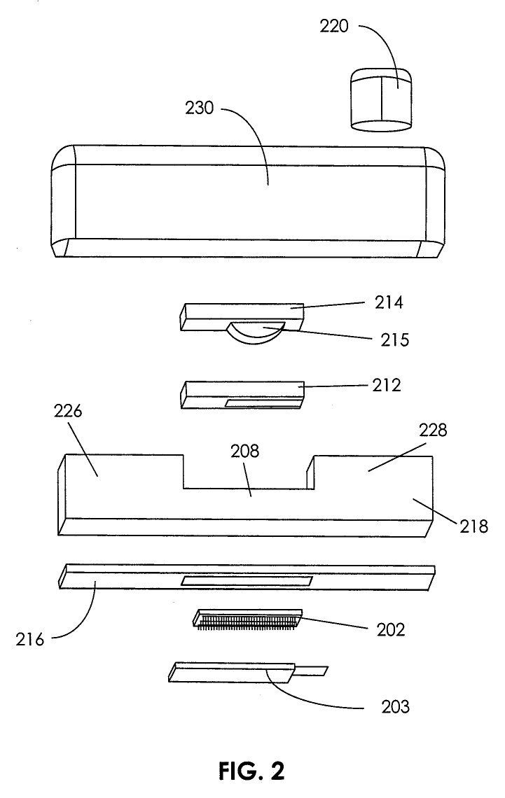

FIG. 2 shows an exploded view of another embodiment of the invention. This figure shows a removable seal 203 covering the sharp distal ends of microneedles 202 and attached, e.g., by adhesive. Seal 203 maintains the sensing fluid within the microneedles and sensing area prior to use and is removed prior to placing the glucose monitor 200 on the skin using adhesive pressure seal 216. In this embodiment, microneedles 202, sensing fluid and waste reservoirs 218 and 226, sensing microchannel 208 and electrochemical glucose sensor 212 are contained within and/or supported by a housing 228 which forms the disposable portion of the device. A second housing 230 supports an electronics board 214 (containing, e.g., processing circuitry, a power source, transmission circuitry, etc.) and an actuator 220 that can be used to move sensing fluid out of reservoir 218, through microchannel 208 into waste reservoir 226. Electrical contacts 215 extend from electronics board 214 to make contact with corresponding electrodes in glucose sensor 212 when the device is assembled.

The following is a description of glucose sensors that may be used with the glucose monitors of this invention. In 1962 Clark and Lyons proposed the first enzyme electrode (that was implemented later by Updike and Hicks) to determine glucose concentration in a sample by combining the specificity of a biological system with the simplicity and sensitivity of an electrochemical transducer. The most common strategies for glucose detection are based on using either glucose oxidase or glucose dehydrogenase enzyme.

Electrochemical sensors for glucose, based on the specific glucose oxidizing enzyme glucose oxidase, have generated considerable interest. Several commercial devices based on this principle have been developed and are widely used currently for monitoring of glucose, e.g., self testing by patients at home, as well as testing in physician offices and hospitals. The earliest amperometric glucose biosensors were based on glucose oxidase (GOX) which generates hydrogen peroxide (H₂O₂) in the presence of oxygen and glucose according to the following reaction scheme:

Glucose+GOX-FAD (ox)→Gluconolactone+GOX-FADH₂ (red)

GOX-FADH₂ (red)+O₂→GOX-FAD (ox)+H₂O₂

Electrochemical biosensors are used for glucose detection because of their high sensitivity, selectivity and low cost. In principal, amperometric detection is based on measuring either the oxidation or reduction of an electroactive compound at a working electrode (sensor). A constant potential is applied to that working electrode with respect to another electrode used as the reference electrode. The glucose oxidase enzyme is first reduced in the process but is reoxidized again to its active form by the presence of any oxygen resulting in the formation of hydrogen peroxide. Glucose sensors generally have been designed by monitoring either the hydrogen peroxide formation or the oxygen consumption. The hydrogen peroxide produced is easily detected at a potential of +0.6 V relative to a reference electrode such as a silver/silver chloride electrode. However, sensors based on hydrogen peroxide detection are subject to electrochemical interference by the presence of other oxidizable species in clinical samples such as blood or serum. On the other hand, biosensors based on oxygen consumption are affected by the variation of oxygen concentration in ambient air. In order to overcome these drawbacks, different strategies have been developed and adopted.

Selectively permeable membranes or polymer films have been used to suppress or minimize interference from endogenous electroactive species in biological samples. Another strategy to solve these problems is to replace oxygen with electrochemical mediators to reoxidize the enzyme. Mediators are electrochemically active compounds that can reoxidize the enzyme (glucose oxidase) and then be reoxidized at the working electrode as shown below:

GOX-FADH₂ (red)+Mediator (ox)→GOX-FAD (ox)+Mediator (red)

Organic conducting salts, ferrocene and ferrocene derivatives, ferricyanide, quinones, and viologens are considered good examples of such mediators. Such electrochemical mediators act as redox couples to shuttle electrons between the enzyme and electrode surface. Because mediators can be detected at lower oxidation potentials than that used for the detection of hydrogen peroxide the interference from electroactive species (e.g., ascorbic and uric acids present) in clinical samples such as blood or serum is greatly reduced. For example ferrocene derivatives have oxidation potentials in the +0.1 to 0.4 V range. Conductive organic salts such as tetrathiafulvalene-tetracyanoquinodimethane (TTF-TCNQ) can operate as low as 0.0 Volts relative to a silver/silver chloride reference electrode. Nankai et al, WO 86/07632, published Dec. 31, 1986, discloses an amperometric biosensor system in which a fluid containing glucose is contacted with glucose oxidase and potassium ferricyanide. The glucose is oxidized and the ferricyanide is reduced to ferrocyanide. This reaction is catalyzed by glucose oxidase. After two minutes, an electrical potential is applied, and a current caused by the re-oxidation of the ferrocyanide to ferricyanide is obtained. The current value, obtained a few seconds after the potential is applied, correlates to the concentration of glucose in the fluid.

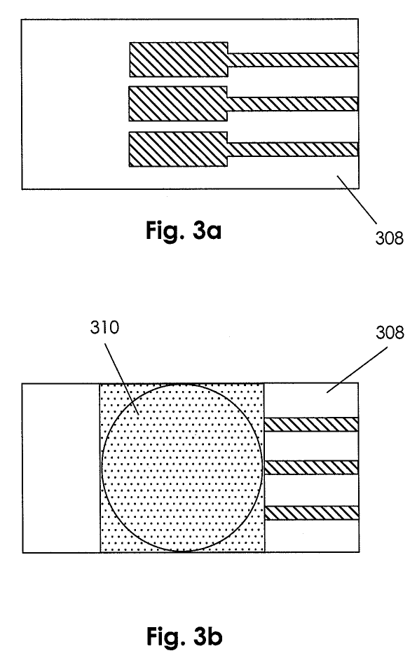

There are multiple glucose sensors that may be used with this invention. In a three electrode system, shown in FIG. 3(a), a working electrode 302 is referenced against a reference electrode 304 (such as silver/silver chloride) and a counter electrode 306 (such as platinum) provides a means for current flow. The three electrodes are mounted on a substrate 308, then covered with a reagent 310, as shown in FIG. 3(b).

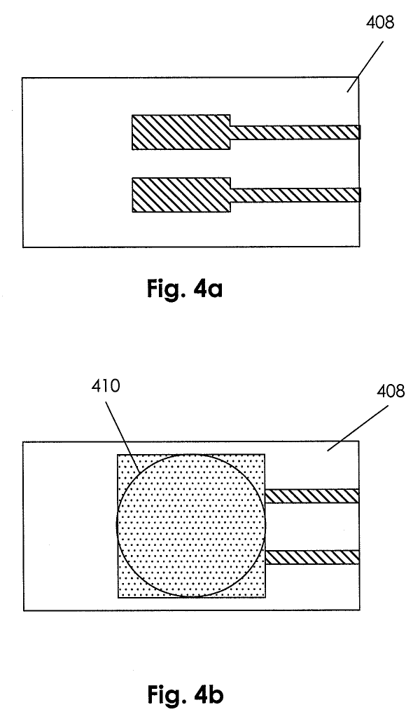

FIG. 4 shows a two electrode system, wherein the working and counter electrodes 402 and 404 are made of different electrically conducting materials. Like the embodiment of FIG. 3, the electrodes 402 and 404 are mounted on a flexible substrate 408 as shown in FIG. 4(a) and covered with a reagent 410, as shown in FIG. 4(b). In an alternative two electrode system, the working and counter electrodes are made of the same electrically conducting materials, where the reagent exposed surface area of the counter electrode is slightly larger than that of the working electrode or where both the working and counter electrodes are substantially of equal dimensions.

In amperometric and coulometric biosensors, immobilization of the enzymes is also very important. Conventional methods of enzyme immobilization include covalent binding, physical adsorption or cross-linking to a suitable matrix may be used.

In some embodiments, the reagent is contained in a reagent well in the biosensor. The reagent includes a redox mediator, an enzyme, and a buffer, and covers substantially equal surface areas of portions of the working and counter electrodes. When a sample containing the analyte to be measured, in this case glucose, comes into contact with the glucose biosensor the analyte is oxidized, and simultaneously the mediator is reduced. After the reaction is complete, an electrical potential difference is applied between the electrodes. In general the amount of oxidized form of the redox mediator at the counter electrode and the applied potential difference must be sufficient to cause diffusion limited electrooxidation of the reduced form of the redox mediator at the surface of the working electrode. After a short time delay, the current produced by the electrooxidation of the reduced form of the redox mediator is measured and correlated to the amount of the analyte concentration in the sample. In some cases, the analyte sought to be measured may be reduced and the redox mediator may be oxidized.

In the present invention, these requirements are satisfied by employing a readily reversible redox mediator and using a reagent with the oxidized form of the redox mediator in an amount sufficient to insure that the diffusion current produced is limited by the oxidation of the reduced form of the redox mediator at the working electrode surface. For current produced during electrooxidation to be limited by the oxidation of the reduced form of the redox mediator at the working electrode surface, the amount of the oxidized form of the redox mediator at the surface of the counter electrode must always exceed the amount of the reduced form of the redox mediator at the surface of the working electrode. Importantly, when the reagent includes an excess of the oxidized form of the redox mediator, as described below, the working and counter electrodes may be substantially the same size or unequal size as well as made of the same or different electrically conducting material or different conducting materials. From a cost perspective the ability to utilize electrodes that are fabricated from substantially the same material represents an important advantage for inexpensive biosensors.

As explained above, the redox mediator must be readily reversible, and the oxidized form of the redox mediator must be of sufficient type to receive at least one electron from the reaction involving enzyme, analyte, and oxidized form of the redox mediator. For example, when glucose is the analyte to be measured and glucose oxidase is the enzyme, ferricyanide or quinone may be the oxidized form of the redox mediator. Other examples of enzymes and redox mediators (oxidized form) that may be used in measuring particular analytes by the present invention are ferrocene and or ferrocene derivative, ferricyanide, and viologens. Buffers may be used to provide a preferred pH range from about 4 to 8. The most preferred pH range is from about 6 to 7. The most preferred buffer is phosphate (e.g., potassium phosphate) from about 0.1M to 0.5M and preferably about 0.4M. (These concentration ranges refer to the reagent composition before it is dried onto the electrode surfaces.) More details regarding glucose sensor chemistry and operation may be found in: Clark L C, and Lyons C, “Electrode Systems for Continuous Monitoring in Cardiovascular Surgery,” Ann NY Acad Sci, 102:29, 1962; Updike S J, and Hicks G P, “The Enzyme Electrode,” Nature, 214:986, 1967; Cass, A. E. G., G. Davis. G. D. Francis, et. al. 1984. Ferrocene-mediated enzyme electrode for amperometric determination of glucose. Anal. Chem. 56:667-671; and Boutelle, M. G., C. Stanford. M. Fillenz et. al. 1986. An amperometric enzyme electrode for monitoring brain glucose in the freely moving rat. Neurosci lett. 72:283-288.

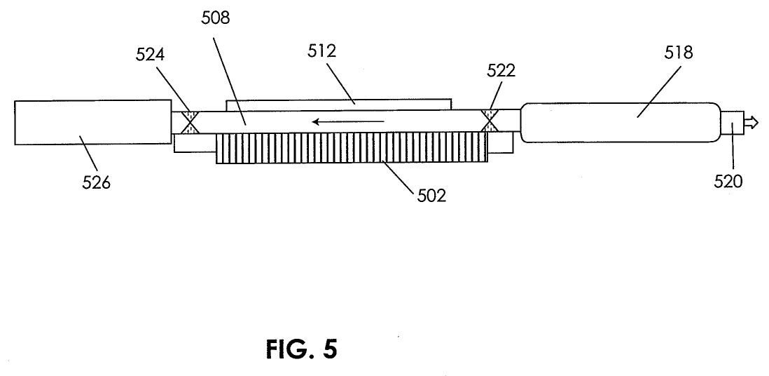

Another embodiment of the disposable portion of the glucose monitor invention is shown in FIG. 5 with a microneedle array 502 and a glucose sensor 512 in fluid communication with a sensing area in channel 508. In this embodiment, actuator 520 is on the side of sensing fluid reservoir 518, and the waste reservoir 526 is expandable. Operation of actuator 520 sends sensing fluid from reservoir 518 through one way flap valve 522 into the sensing area in channel 508 and forces sensing fluid within channel 508 through flap valve 524 into the expandable waste reservoir 526.

In the embodiment of FIG. 5 (and potentially other embodiments), the starting amount of sensing fluid in the calibration reservoir 518 is about 1.0 ml or less, and operation of the sensing fluid actuator 520 sends a few microliters (e.g., 10 μL) of sensing fluid into channel 508. Recalibrating the device three times a day for seven days will use less than 250 μL of sensing fluid.

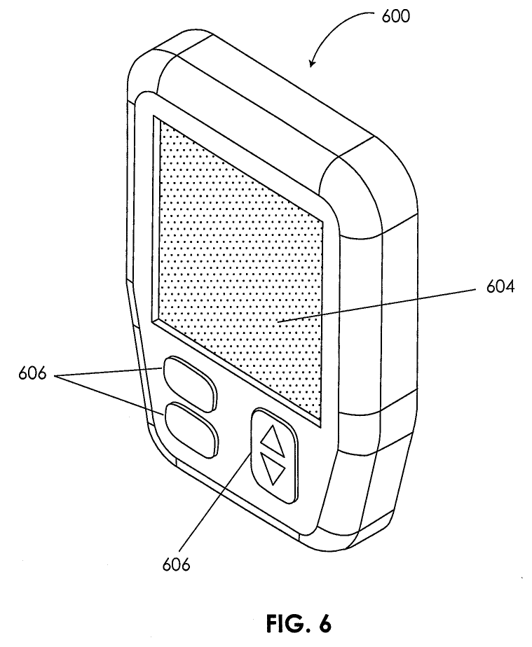

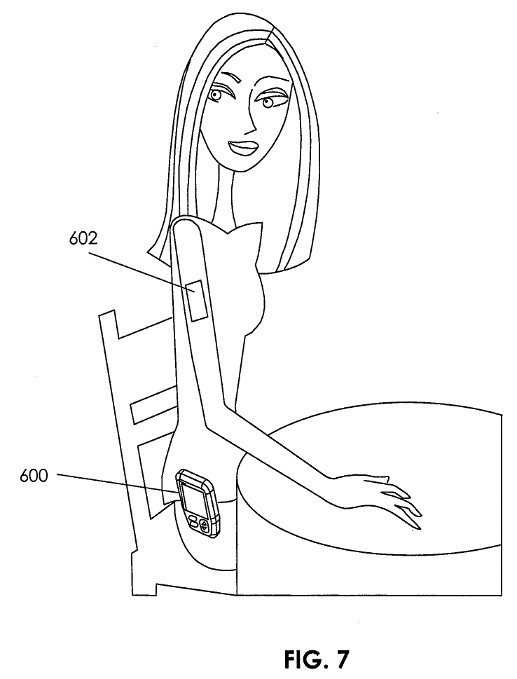

FIGS. 6 and 7 show a remote receiver for use with a glucose monitoring system. The wireless receiver can be configured to be worn by a patient on a belt, or carried in a pocket or purse. In this embodiment, glucose sensor information is transmitted by the glucose sensor 602 applied to the user's skin to receiver 600 using, e.g., wireless communication such as radio frequency (RF) or Bluetooth wireless. The receiver may maintain a continuous link with the sensor, or it may periodically receive information from the sensor. The sensor and its receiver may be synchronized using RFID technology or other unique identifiers. Receiver 600 may be provided with a display 604 and user controls 606. The display may show, e.g., glucose values, directional glucose trend arrows and rates of change of glucose concentration. The receiver can also be configured with a speaker adapted to deliver an audible alarm, such as high and low glucose alarms. Additionally, the receiver can include a memory device, such as a chip, that is capable of storing glucose data for analysis by the user or by a health care provider.

In some embodiments, the source reservoir for the calibration and sensing fluid may be in a blister pack which maintains its integrity until punctured or broken. The actuator may be a small syringe or pump. Use of the actuator for recalibration of the sensor may be performed manually by the user or may be performed automatically by the device if programmed accordingly. There may also be a spring or other loading mechanism within the reusable housing that can be activated to push the disposable portion—and specifically the microneedles—downward into the user's skin.

### Sensing Cycle of the Glucose Sensor

The glucose sensor may be operated continuously with respect to the sensing operation of the glucose sensor. In some embodiments, the glucose diffuses through the fluid in the needle lumens of the microneedle array 102 to the electrode surface. The glucose reacts with the chemistry shown above (i.e., paragraphs 0041 and 0042) to produce H₂O₂. The H₂O₂ is then detected in one continuous process. A sensor operating continuously may measure a smaller signal, but likely a more stable signal (which would slowly change as the blood glucose level changes) as compared to a sensor operating periodically/intermittently. When the glucose sensor is operated continuously, the electrodes are likely to be biased and may be kept biased continuously. The glucose sensor may be operated continuously until calibration.

The glucose sensor may also be operated periodically or intermittently. Periodic operation involves a sensing cycle with regular timing. Periodic operation may occur when the glucose sensor is turned on and off (i.e., when the electrodes are biased and not biased) according to some regular schedule. An example of a regular schedule may be 15 minutes out of every 30 minutes. Periodic sensor operation would allow detection of a larger signal over the shorter times the sensor is activated (and therefore, potentially a better signal to noise ratio).

Intermittent operation involves a sensing cycle that does not require a regular timing. Intermittent operation may occur when the glucose sensor is turned on and off (i.e., when the electrodes are biased and not biased), but not necessarily in a regular cycle. For example, the user may push a button to initiate an intermittent glucose sensing cycle. Initiation of the glucose sensing cycle may also be prompted by other events (i.e., before or after meals). Intermittent sensor operation may also give discrete readings at some measurement interval (minutes). Intermittent sensor operation may also occur at specific times of the day.

Any of these types of sensing cycles (i.e., continuous, periodic and intermittent) may involve averaging of signals.

An example of a sensing cycle is outlined below. Glucose continuously diffuses through the microneedle array 102 into a sensing volume. The glucose sensor may be turned on (or may already be on). As more glucose diffuses in, the H₂O₂ concentration increases. At some point, the electrodes are biased, the entire volume of H₂O₂ is detected coulometrically and its concentration depleted down to substantially zero. Further examples of “sensing to depletion” may be found in U.S. Pat. Nos. 6,299,578 and 6,309,351. Equilibrium (i.e., the concentration of glucose in the chamber is equal to the concentration of glucose in the tissue) does not necessarily need to be achieved. Furthermore, the level of glucose in the chamber does not necessarily need to be at a constant state during the measurement cycle. Additionally, the sensing volume does not necessarily need to be flushed after the glucose is depleted. The timing of when to bias the electrode(s) may be dependent on the type of sensing cycle, and may need to be determined empirically. For example, if a periodic sensing scheme were used, the timing of when to bias the electrodes would be part of the timing of the sensing period. In addition, when the glucose sensor is turned on (or may already be on) and is depleting the H₂O₂, new H₂O₂ is being formed as glucose reacts with the GOx enzyme.

### Geometry of the Glucose Sensor

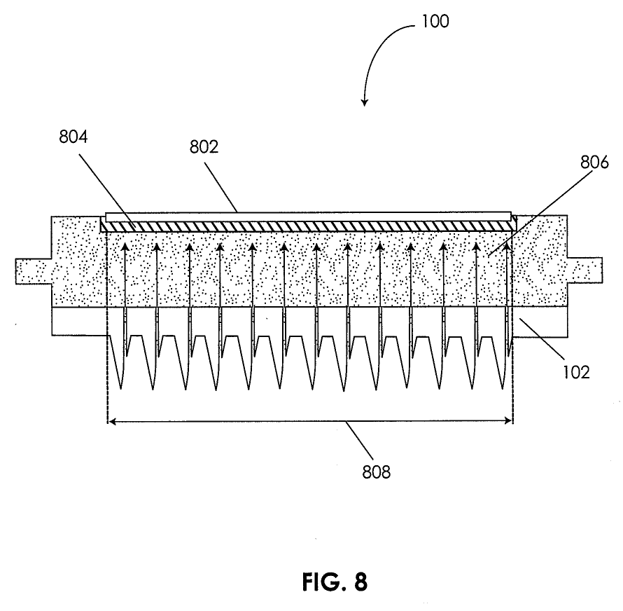

FIG. 8 shows another schematic cross-section of the glucose monitor 100. The glucose monitor 100 includes a microneedle array chip (MAC) 102, working electrode 802 (glucose sensor) based on glucose oxidase (GOX) chemistry 804 and sensing volume 806. FIG. 8 shows an example of desirable geometry 808 of the working electrode 802, sensing volume 806 and microneedle array 102. In this example, the area of the working electrode 802 is similar to or slightly larger than the area of microneedle array 102. The working electrode area should approximate the area (and shape) of the microneedle array 102. In some embodiments, the area of the working electrode may be in the range of 10 mm² to 100 mm². One example of the working electrode area is 5.5 mm×5.5 mm, or 30.25 mm². An example of the working electrode 802 geometry is a planar electrode that is slightly larger than the microneedle array 102. Another example of the working electrode 802 geometry is a closely spaced electrode strip (as depicted in U.S. Pat. No. 6,139,718). Other examples include electrodes with a similar effective area and which detect a similar sensing volume as sensing volume 806.

In order to efficiently measure the glucose that is collected through the microneedle array 102, the area of the working electrode 802 should approximate the area of the microneedle array 102 and the working electrode 802 should be located behind the microneedle array 102. As shown in FIG. 8, the working electrode 802 may be located on one side of the sensing volume 806 and on the opposite side of the microneedle array 102.

On the other hand, if the working electrode 802 area were much smaller than the area of the microneedle array 102, there would be appreciable glucose collected outside the perimeter of the working electrode 802. The time necessary for this glucose to diffuse to the working electrode 802 may be longer. A time lag to measure this glucose may then result. A lag time between interstitial fluid glucose and the measured glucose value may also result.

In FIG. 8, the thickness of the sensing volume 806 is as small as possible to reduce the distance that glucose must diffuse through the sensing volume 806. Accordingly, the diffusion path from the microneedle array 102 to the working electrode 802 is as short as possible as indicated by the vertical arrows. In some embodiments, the thickness of the sensing volume 806 is in range of about 50 microns to about 3000 microns. In other embodiments, the thickness is between about 50 microns to about 500 microns.

The thickness of the sensing volume and 806, therefore, its total volume, has effects on the sensing characteristics. As the thickness of the sensing volume is decreased, the diffusion distance and the diffusion time is decreased, thus decreasing the measurement lag time. For the intermittent sensor operation, the smaller volume results in higher glucose concentration in the sensing volume 806.

The glucose sensor may also include a reference electrode (for a two-electrode system) or a combination of reference and counter electrodes (for a three-electrode system) for proper operation of a sensor. The reference and counter electrodes should be placed in fluid communication with the sensing volume 806 and the working electrode 802. For example, the reference and/or counter electrodes (not shown) may be placed in a co-planner manner with the working electrode, but should be placed outside the desirable geometry 808, as shown in FIG. 8 and described above.

### Continuous Glucose Monitoring

As noted earlier, direct fluid communication occurs between the interstitial fluid, the microneedle lumens, and the sensing volume 806. A constant concentration gradient from the interstitial fluid to the glucose sensor causes diffusion of glucose to occur continuously from the interstitial fluid to the electrode surface. The diffusion may occur continuously without interruption. Accordingly, continuous glucose monitoring occurs over time. While this application refers to continuous glucose monitoring, actual glucose sensing may be continuous, periodic or intermittent, or a combination thereof.

### Calibration of the Glucose Monitor

As noted earlier, calibration may also be performed by the glucose monitor 100 automatically without any input from the user. In some embodiments, the sensing (calibration) fluid containing a known concentration of glucose is delivered into the sensing volume 806 and sensed by the glucose sensor. This calibration corrects for any drift in the intrinsic sensor sensitivity over time and may be performed automatically by the device. This intrinsic sensor sensitivity is the amount of sensor signal generated for a given glucose concentration in the sensing volume 806. The overall sensitivity of the glucose monitor device is the amount of sensor signal generated for a given blood glucose concentration. The overall sensitivity of the system may be a function of both how much glucose is collected through the microneedles and the sensitivity of the sensor.

The calibration scheme calibrates the intrinsic sensor sensitivity as the microneedle array 102 is bypassed by delivering the calibration fluid directly into the sensing volume 806. The intrinsic sensor sensitivity of the sensor may drift over time because of changes in the electrode surface, poisoning of the platinum catalyst on the surface, or adsorption of other chemical species (e.g., proteins) collected through the needles. The intrinsic sensor sensitivity of the sensor may drift for other reasons as well.

In some embodiments of the invention, the rate of transport of the glucose from the interstitial fluid to the sensor is constant each time the glucose monitor 100 is used and thus, does not have to be calibrated.

In addition, multiple calibration fluids may be utilized. These multiple calibration fluids may or may not have different amounts of buffers, preservatives or other components in addition to glucose.

Using one calibration fluid, a one-point calibration is performed. The one-point calibration may assume an intercept of the calibration curve is zero (or assume some other empirically determined value). The one-point calibration may also adjust the slope of the calibration curve. If two calibration fluids with different glucose concentrations are utilized, an intercept value may not need to be assumed. The best-fit calibration curve may be determined from the sensor signals generated by two different glucose concentrations.

Calibration may occur in a variety of ways. Calibration may occur with respect to time such as at a predetermined time (or predetermined times) or at a predetermined time interval. Calibration may also occur when the glucose monitor 100 detects drifts in the sensor signal. Drifts in the sensor signal may be determined by monitoring the sensor signal and looking for any excursions that could not be caused by normal glucose level movement or diffusion. Examples of such drifts may be discontinuities in the sensor signal, sharp sensor changes, high noise levels, etc. In addition, calibration may also occur in response to an event or occur at any predetermined points that may or may not be time associated.

The steps that occur during the calibration process of one exemplary embodiment are detailed below. The sensing (calibration) fluid flows into the sensing volume 806. The sensor is activated or the sensor may already be activated. A sensor signal is acquired that indicates the concentration of glucose in the sensing fluid. The sensing operation may continue for a length of time to acquire the sensor signal. However, the sensing operation should not continue for a length of time such that an appreciable amount of glucose diffuses into the sensing volume 806 from the microneedle array 102. The sensing operation may also continue for a length of time sufficient to deplete the concentration of glucose in the sensing fluid down to the amount of the glucose in the sensing fluid that had originally flowed into the sensing volume 806. The sensing fluid remains in the sensing volume 806 and glucose diffuses from the microneedle array 102 into the sensing fluid.

The glucose monitor 102 may use an algorithm that uses a measure of the intrinsic sensor sensitivity or the overall sensitivity of the system from the calibration process to make adjustments on the measured glucose concentration diffusing into the sensing volume 806 through the microneedle array 102. As an example, a known glucose concentration may flow into the sensing volume 806 and a sensor signal may be acquired. Accordingly, the sensor signal may be used to make adjustments on the measurement (i.e., continuous measurement) of glucose diffusing into the sensing volume 806. For example, if the previous calibration had generated a sensitivity of “100”, and the most recent calibration generates a sensitivity of “95”, then it would indicate a loss of sensitivity of the system. The values displayed to the user for glucose collected through the microneedle array 102 would be reading lower than the true value, and would have to be adjusted upwards an amount related to the change in the calibration values to correct for this.

As noted earlier, the concentration of glucose in the sensing (calibration) fluid is described in the range from 0 to 400 mg/dL. This concentration range is the possible glucose concentrations that could be measured by the device. The concentration of glucose in the sensing volume 806 (when glucose measurements are taken) may be lower than the interstitial glucose concentration because the microneedle array 102 has such a small cross-sectional diffusion area and because the sensor may be continuously operating and depleting the glucose while sensing it. Therefore, the concentration of the glucose in the sensing (calibration) fluid is likely to be on the order of magnitude of the concentration of glucose that is in the sensing volume 806 while the device is operating in a non-calibration mode (i.e., measuring the glucose diffusing through the microneedles). This concentration may then be on the order of micromolar to millimolar (i.e., 1-3 orders of magnitude lower than the average 100 mg/dL (5.5 mM) blood glucose concentration).

### Empty Needles

One embodiment of the glucose monitor 100 includes microneedle array 102 having microneedles that are pre-filled with sensing fluid prior to the use of the device. Another embodiment of the glucose monitor 100 includes microneedles that are not pre-filled prior to the use of the device. In this embodiment, the microneedle lumens may be filled with the interstitial fluid once the array 102 is applied to the skin. Glucose may then diffuse from the body's interstitial fluid through the microneedle lumens and into the sensing volume 806.

The interstitial fluid may flow immediately into the lumens of the microneedles upon insertion of unfilled needles. Capillary action may fill the lumens with interstitial fluid.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, the devices, systems and methods described above may be used to monitor analytes other than glucose. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A glucose monitor comprising:
a plurality of tissue piercing elements, each tissue piercing element comprising a distal opening,
a proximal opening and an interior space extending between the distal and proximal openings;
a sensing volume in fluid communication with the proximal openings of the tissue piercing elements;
sensing fluid extending into the sensing volume; and
a glucose sensor adapted to detect a concentration of glucose in the sensing fluid within the sensing volume.

2. The glucose monitor of claim 1 wherein the glucose sensor is an electrochemical sensor.

3. The glucose monitor of claim 1 wherein an area of a surface that faces the tissue piercing elements of the glucose sensor is substantially similar to an area covering the tissue piercing elements.

4. The glucose monitor of claim 1 wherein an area of a surface that faces the tissue piercing elements of the glucose sensor is larger than the area covering the tissue piercing elements.

5. The glucose monitor of claim 1 wherein an area of a surface that faces the tissue piercing elements of the glucose sensor is in the range of 10 mm² to 100 mm².

6. The glucose monitor of claim 1 wherein a thickness of the sensing volume is in the range of 50 microns to 3000 microns.

7. The glucose monitor of claim 1 wherein the glucose sensor is adapted to detect a concentration of glucose in the sensing fluid within the sensing volume without extracting interstitial fluid.

8. The glucose monitor of claim 1 wherein the sensing fluid comprises multiple calibration fluids.

9. The glucose monitor of claim 1 wherein the glucose sensor is configured to operate continuously.

10. The glucose monitor of claim 1 wherein the glucose sensor is configured to operate periodically.

11. The glucose monitor of claim 1 wherein the glucose sensor is configured to operate intermittently.

12. A method of in vivo monitoring of an individual's interstitial fluid glucose concentration comprising:
inserting distal ends of a plurality of tissue piercing elements through a stratum corneum area of the individual's skin, the tissue piercing elements each comprising a distal opening, a proximal opening, an interior space extending between the distal and proximal openings, and a sensing fluid filling substantially the entire interior space;
allowing glucose to diffuse into a sensing volume without extracting interstitial fluid; and
sensing a glucose concentration of the sensing fluid within the sensing volume.

13. The method of claim 12 wherein sensing the glucose concentration further comprises continuing to monitor the glucose concentration over time.

14. The method of claim 12 wherein sensing the glucose concentration comprises continuously sensing the glucose concentration over time.

15. The method of claim 14 wherein continuous sensing of the glucose concentration proceeds until calibration.

16. The method of claim 13 wherein sensing the glucose concentration comprises periodically sensing the glucose concentration.

17. The method of claim 16 wherein periodically sensing the glucose concentration comprises having a sensing cycle with regular timing.

18. The method of claim 13 wherein sensing the glucose concentration comprises intermittently sensing the glucose concentration.

19. The method of claim 18 wherein intermittently sensing the glucose concentration comprises a sensing cycle having irregular timing.

20. The method of claim 12 wherein a glucose sensor senses the glucose concentration, the method further comprising calibrating the glucose sensor prior to the sensing step.

21. The method of claim 20 wherein the calibrating step occurs at a predetermined time point.

22. The method of claim 20 wherein the calibrating step occurs at a predetermined time interval.

23. The method of claim 20 wherein the calibrating step occurs when the glucose sensor detects a drift in the glucose concentration measurement.

24. The method of claim 23 wherein the drift is determined by monitoring a sensor signal from the glucose sensor.

25. The method of claim 20 wherein the calibrating step comprises moving the sensing fluid into the sensing volume.

26. The method of claim 25 wherein the calibrating step further comprises acquiring a sensor signal indicating the concentration of glucose in the sensing fluid.

27. The method of claim 26 further comprising moving sensing fluid out of the sensing area as sensing fluid is moved into the sensing volume.

28. The method of claim 27 wherein the sensing fluid remains in the glucose sensor after the calibrating step.

29. The method of claim 27 wherein the step of moving sensing fluid comprises moving sensing fluid having a glucose concentration of between about 0 mg/dl and about 400 mg/dl.

30. The method of claim 12 wherein sensing a glucose concentration comprises:
diffusing glucose through the tissue piercing elements; and
detecting hydrogen peroxide formation.

31. The method of claim 30 further comprising detecting hydrogen peroxide formation coulometrically.

32. The method of claim 30 wherein the hydrogen peroxide formation is reduced to substantially zero.

33. The method of claim 12 wherein sensing a glucose concentration comprises:
diffusing glucose through the tissue piercing elements; and
detecting oxygen consumption.

34. A method of in vivo monitoring of an individual's interstitial fluid glucose concentration comprising:
inserting distal ends of a plurality of tissue piercing elements through a stratum corneum area of the individual's skin, the tissue piercing elements each comprising a distal opening, a proximal opening, and an interior space extending between the distal and proximal opening;
allowing interstitial fluid to flow into the interior space of the tissue piercing elements to substantially fill the interior space;
filling substantially the entire interior space of the sensing area with sensing fluid; and
sensing a glucose concentration of the sensing fluid.

35. The method of claim 34 wherein the interstitial fluid does not flow past the proximal opening.

36. The method of claim 34 wherein the interstitial fluid flows immediately into the interior space of the tissue piercing elements.
